(19) United States
(12) Patent Application Publication
(10) Pub. No.: US 2021/0129189 A1
(43) Pub. Date: May 6, 2021

(54) EARPHONE STORAGE DEVICE

(71) Applicant: SHENZHEN CANNICE TECHNOLOGY CO., LTD., Shenzhen (CN)

(72) Inventors: Xiayun GUO, Shenzhen (CN); Yu-Cheng LIU, Shenzhen (CN)

(73) Assignee: SHENZHEN CANNICE TECHNOLOGY CO., LTD., Shenzhen (CN)

(21) Appl. No.: 17/020,849

(22) Filed: Sep. 15, 2020

(30) Foreign Application Priority Data
Oct. 30, 2019 (CN) 201911046882.4

(51) Int. Cl. B08B 1/00; A61L 2/18; A61L 2/00

(52) U.S. Cl. CPC B08B 1/002; A61L 2/18; A61L 2202/20; A61L 2202/17; A61L 2/0029

(57) ABSTRACT

An earphone storage device includes a charging storage box. A top cover that can be closed is provided on the charging storage box. A storage tank and a cleaning device are provided in the charging storage box, and a charging assembly is provided at the bottom of the storage tank. The cleaning device includes a dust removal assembly, a disinfection assembly and a sterilization assembly. A rotating shaft and a rotating motor are rotatably provided at the center of the top cover. The rotating motor is connected with the rotating shaft, the rotating shaft is connected with a rotating disc, and the rotating disc is provided with three mounting grooves. The dust removal assembly, the disinfection assembly and the sterilization assembly are respectively fixed in the three mounting grooves. When the top cover is closed, the storage tank is located directly below a rotation track of the mounting groove.

### CROSS-REFERENCE TO RELATED APPLICATION

The present application claims the priority to Chinese patent application No. 201911046882.4, titled “EARPHONE STORAGE DEVICE” and filed with the China National Intellectual Property Administration on Oct. 30, 2019, which is incorporated herein by reference.

### FIELD

The present application relates to the technical field of electronic devices, and in particular to an earphone storage device.

### BACKGROUND

With the development of electronic technology, smart wearable devices such as smart earphones, smart watches and bracelets have come out and become more and more popular. The smart earphone needs a storage box to store when not working, and the storage box has the function of charging the smart earphone. The earphone storage box generally has the functions of storing the earphones and charging the earphones. However, when people wear earphones or other various in-ear devices, especially when they wear them frequently, a lot of bacteria will be generated due to the closed environment of the ear cavity. However, according to the current earphone product design, it is impossible to clean the in-ear part. As time passes, more and more bacteria will be accumulated in the in-ear part of the earphone, and the human body will produce a series of adverse reactions, such as itching, pain and other symptoms caused by infection of the ear canal.

Since oil stains, ear canal secretions and other contaminants are often adhered to the earphones when the earphones are in use, the earphones become a breeding ground for bacteria. If these contaminants are not removed, bacteria can easily breed again. However, the existing earphone cleaning device often adopts the ultraviolet sterilization method which cannot remove the above-mentioned contaminants, and the earphones cannot be completely cleaned.

### SUMMARY

An earphone storage device is provided according to the present application to solve the problem that the contaminants attached to the earphones cannot be removed and the earphones cannot be completely cleaned because in the prior art the only method used to remove the contaminants is the ultraviolet sterilization method.

In order to achieve the above object, the following technical solutions are provided according to the present application.

An earphone storage device includes a charging storage box. A top cover that can be closed is provided on the charging storage box. A storage tank and a cleaning device are provided in the charging storage box, and a charging assembly is provided at the bottom of the storage tank. The cleaning device includes a dust removal assembly, a disinfection assembly and a sterilization assembly.

A rotating shaft and a rotating motor are rotatably provided at the center of the top cover. The rotating motor is connected with the rotating shaft, the rotating shaft is connected with a rotating disc, and the rotating disc is provided with three mounting grooves. The dust removal assembly, the disinfection assembly and the sterilization assembly are respectively fixed in the three mounting grooves. When the top cover is closed, the storage tank is located directly below a rotation track of the mounting groove.

Preferably, the dust removal assembly includes a dust removal base. A dust removal rotating shaft is rotatably provided at the center of the dust removal base. The dust removal rotating shaft is connected with a dust removal disc, multiple dust removal strips are radially arranged on the upper surface of the dust removal disc, and each of the dust removal strips is provided with a dust removal brush. The dust removal disc is provided with multiple air jet holes and multiple air suction holes. The air jet holes and the air suction holes are arranged at intervals.

Preferably, the air jet holes are arranged obliquely and have different degrees of inclination, so that air discharge directions are different. The air suction holes are arranged obliquely and have different degrees of inclination, so that air suction directions are different.

Preferably, the dust removal rotating shaft is radially provided with a dust removal rotating rod. The dust removal base is provided with four dust removal driving rods. The dust removal driving rods are distributed around the dust removal rotating shaft in a cross shape. The dust removal rotating rod is provided with a magnetic block, and each of the dust removal driving rods is provided with an electromagnet.

Preferably, the dust removal base is provided with an air suction fan and an air jet fan. A dust collecting box is connected to an air outlet of the air suction fan, an air inlet of the air suction fan is connected to the air suction holes through hoses, and an air outlet of the air jet fan is connected to the air jet holes through hoses.

Preferably, the disinfection assembly includes a disinfection base, and a disinfection rotating shaft is rotatably provided at the center of the disinfection base. The disinfection rotating shaft is connected with a disinfection disc, the disinfection disc is provided with multiple disinfection holes and multiple drying holes, and the disinfection holes and the drying holes are arranged at intervals.

Preferably, the disinfection holes are connected with disinfection nozzles. The disinfection nozzles are arranged obliquely and have different degrees of inclination, so that spraying directions of the disinfectant are different. The drying holes are arranged obliquely and have different degrees of inclination, so that the air discharge directions are different.

Preferably, a disinfection rotating motor is provided on the disinfection base. The disinfection rotating shaft is provided with a micro sprayer and a drying fan. The disinfection rotating motor is connected with the disinfection rotating shaft. A liquid inlet of the micro sprayer is connected with a disinfectant tank, a liquid outlet of the micro sprayer is connected with the disinfection holes through hoses. An air outlet of the drying fan is connected with the drying holes through hoses.

Preferably, the sterilization assembly includes a sterilization base, and a sterilization rotating shaft is rotatably provided at the center of the sterilization base. The sterilization rotating shaft is connected with a sterilization table. The sterilization table is provided with multiple sterilization lamps with different inclination angles. The sterilization base is provided with a sterilization rotating motor, and the sterilization rotating motor is connected with the sterilization rotating shaft.

Preferably, a central control device is further provided in the storage box, and the charging assembly and the cleaning device are both connected with the central control device. After the charging assembly starts working, the central control device drives the cleaning device to operate.

The embodiments of the present application have the following advantages:

The present application retains the storage and charging functions of the existing storage box. Moreover, by providing the cleaning device, the earphones can be dedusted, disinfected and sterilized. The solid dust and oil stains on the earphones are first removed, eliminating the breeding ground of the bacteria. Then, the earphones are sterilized with ultraviolet rays, so that the earphones can be cleaned thoroughly.

### BRIEF DESCRIPTION OF THE DRAWINGS

In order to more clearly illustrate the technical solution in the embodiments of the present application or in the prior art, the drawings referred to in the description of the embodiments or the prior art will be briefly introduced hereinafter. Obviously, the drawings in the following description are only some embodiments of the present application. For those skilled in the art, other drawings can also be obtained according to these drawings without any creative work.

FIG. 1 is a schematic view of the overall structure of an embodiment of the present application;

FIG. 2 is a schematic view of a dust removal assembly in an embodiment of the present application;

FIG. 3 is a schematic plan view of a dust removal disc in an embodiment of the present application;

FIG. 4 is a schematic structural view of a dust removal driving rod in an embodiment of the present application;

FIG. 5 is a schematic structural view of a disinfection assembly in an embodiment of the present application; and

FIG. 6 is a schematic structural view of a sterilization assembly in an embodiment of the present application.

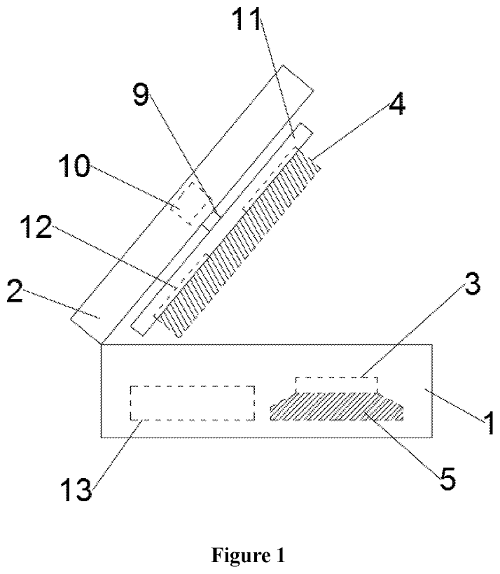

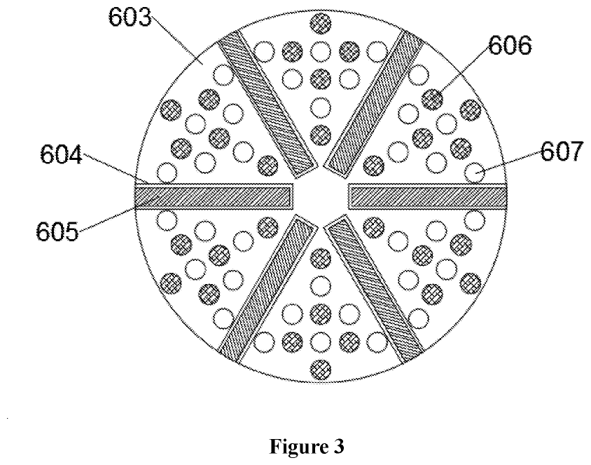

Reference numerals in FIGS. 1 to 6 are listed as follows:

| | |
|---|---|
| 1 charging storage box; | 2 top cover; |
| 3 storage tank; | 4 cleaning device; |
| 5 charging assembly; | 6 dust removal assembly; |
| 7 disinfection assembly; | 8 sterilization assembly; |
| 9 rotating shaft; | 10 rotating motor; |
| 11 rotating disc; | 12 mounting groove; |
| 13 central control device; | |
| 601 dust removal base; | 602 dust removal rotating shaft; |
| 603 dust removal disc; | 604 dust removal strip; |
| 605 dust removal brush; | 606 air jet hole; |
| 607 air suction hole; | 608 dust removal rotating rod; |
| 609 dust removal driving rod; | 610 magnetic block; |
| 611 electromagnet; | 612 air suction fan; |
| 613 air suction fan; | 614 dust collecting box; |
| 701 disinfection base; | 702 disinfection rotating shaft; |
| 703 disinfection disc; | 704 disinfection hole; |
| 705 drying hole; | 706 disinfection nozzle; |
| 707 disinfection rotating motor; | 708 micro sprayer; |
| 709 drying fan; | 710 disinfection tank; |
| 801 sterilization base; | 802 sterilization rotating shaft; |
| 803 sterilization table; | 804 sterilization lamp; |
| 805 sterilization rotating motor. | |

### DETAILED DESCRIPTION OF THE EMBODIMENTS

For making the objects, features and advantages of the present application clearer and easier to be understood, the technical solutions according to the present application are described in detail hereinafter in conjunction with the drawings in the embodiments of the present application. Apparently, the embodiments described hereinafter are only a part of the embodiments of the present application, rather than all embodiments. Based on the embodiments of the present application, all of other embodiments, made by those skilled in the art without any creative efforts, fall into the scope of the present application.

In the description of the present application, it should be understood that, if a component is considered to be “connected” to another component, the former component may be directly connected to another component or there may be an intermediate component therebetween. If a component is considered to be “arranged on” another component, the former component may be directly arranged on another component or there may be an intermediate component therebetween.

In addition, it should be understood that the orientation or positional relationships indicated by terms “long”, “short”, “inner”, “outer” and the like are based on the orientation or positional relationships shown in the drawings, and are merely for the convenience of describing the present application, and do not indicate or imply that the device or element referred to must be in a particular orientation, or be constructed and operated in a particular orientation, and therefore should not be construed as a limit to the scope of the present application.

Technical solutions of the present application will be illustrated in detail through the embodiments and in conjunction with drawings.

### First Embodiment

As shown in FIGS. 1 to 6, an earphone storage device is provided according to the present application, which includes a charging storage box 1. A top cover 2 that can be closed is provided on the charging storage box 1, and a storage tank 3 and a cleaning device 4 are provided in the charging storage box 1. A charging assembly 5 is provided at the bottom of the storage tank 3. A central control device 13 is further provided in the storage box 1. Both the charging assembly 5 and the cleaning device 4 are connected to the central control device 13. After the charging assembly 5 starts working, the central control device 13 drives the cleaning device 4 to operate. In this embodiment, the storage tank 3 is a groove adapted to the earphones, the earphones can be placed in the storage tank 3 and can be kept fixed, and when the earphones are fixed in the storage tank 3, the earphones are connected to the charging assembly 5 to realize charging.

In this embodiment, the charging assembly 5 adopts the charging technology in the prior art, and does not involve innovation in this aspect, as long as charging of the Bluetooth earphones can be realized, so the specific structure thereof will not be described here.

In this embodiment, the cleaning device 4 includes a dust removal assembly 6, a disinfection assembly 7, and a sterilization assembly 8. A rotating shaft 9 and a rotating motor 10 are rotatably provided at the center of the top cover 2. The rotating motor 10 is connected with the rotating shaft 9, the rotating shaft 9 is connected with a rotating disc 11, and the rotating disc 11 is provided with three mounting grooves 12. The dust removal assembly 6, the disinfection assembly 7 and the sterilization assembly 8 are respectively fixed in the three mounting grooves 12. When the top cover 2 is closed, the storage tank 3 is located directly below a rotation track of the mounting groove 12.

The earphone sterilization device according to this embodiment is used in the following manner. The top cover 2 is opened, then the earphones are placed in the storage tank 3 and fixed, and the earphones can be charged at this time, then the top cover 2 is closed, the rotating motor 10 drives the rotating disc 11 to rotate, so that the dust removal assembly 6 is rotated to a position above the earphones, then the dust removal assembly 6 works to remove the solid dust on the earphones, and after a period of time, the dust removal assembly 6 stops, and the rotating disc 11 is further rotated, so that the disinfection assembly 7 is rotated to the position above the earphones, then the disinfection assembly 7 works to remove oil stains on the earphones and perform preliminary disinfection and sterilization, and after a period of time, the disinfection assembly 7 stops, and the rotating disc 11 is further rotated, so that the sterilization assembly 8 is rotated to the position above the earphones, then the sterilization assembly 8 works to further disinfect and sterilize the earphones, and the disinfection assembly 7 stops after a period of time. Through the above arrangement, the earphones can be cleaned while being stored and charged, and in the whole cleaning process, solid dust and oil stains on the earphones can be removed through the dust removal and disinfection process, and the breeding ground of bacteria is eliminated. In combination with the sterilization process, bacteria on the earphones are removed, so that the earphones are cleaned more thoroughly.

As shown in FIGS. 1 to 4, the dust removal assembly 6 in the present embodiment includes a dust removal base 601. The dust removal base 601 is fixed in one of the mounting grooves 12. A dust removal rotating shaft 602 is rotatably provided at the center of the dust removal base 601. The dust removal rotating shaft 602 is connected with a dust removal disc 603, and multiple dust removal strips 604 are radially arranged on the upper surface of the dust removal disc 603. Each of the dust removal strips 604 is provided with a dust removal brush 605. The dust removal disc 603 is provided with multiple air jet holes 606 and multiple air suction holes 607, and the air jet holes 606 and the air suction holes 607 are arranged at intervals. The dust removal base 601 is provided with an air suction fan 612 and an air jet fan 613. A dust collecting box 614 is connected to an air outlet of the air suction fan 612, an air inlet of the air suction fan is connected to the air suction holes 607 through hoses, and an air outlet of the air j et fan 613 is connected to the air jet holes 606 through hoses.

In this embodiment, the dust removal rotating shaft 602 in the dust removal assembly 6 is rotatable, and is driven by a dust removal driving assembly. When the dust removal rotating shaft 602 rotates, the dust removal brushes 605 continuously sweep over the surface of the earphones, thereby sweeping off the solid dust adhered to the surface of the earphones. In the meantime, the air jet fan 613 and the air suction fan 612 work, so that air flow is blown out from the air jet holes 606 and sucked in from the air suction holes 607. The air flow blown out from the air jet holes 606 blows off the solid dust not brushed off by the dust removal brushes 605 and the solid dust attached to where the dust removal brushes 605 cannot reach, and then the solid dust is sucked in through the air suction holes 607 and collected into the dust collecting box 614.

Through the above arrangement, the dust removal assembly 6 can more thoroughly sweep off and collect the solid dust on the earphones to ensure the dust removal effect.

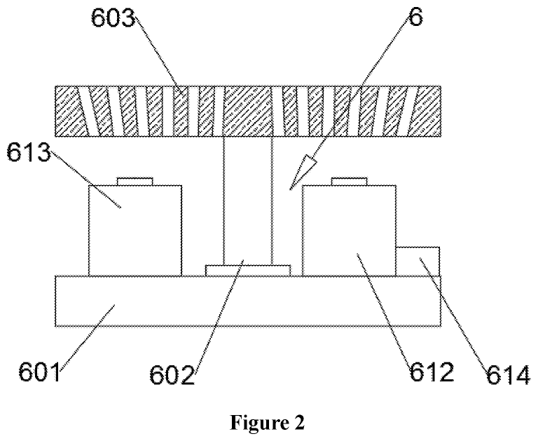

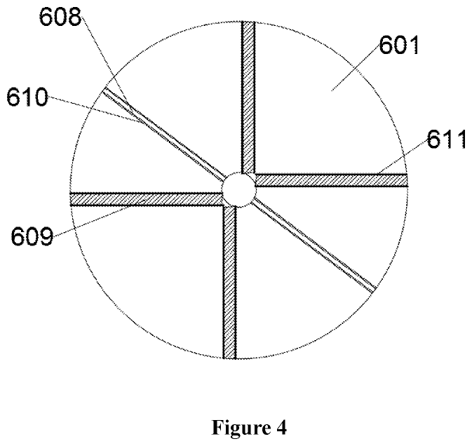

As shown in FIGS. 2 and 4, the dust removal assembly described above includes a dust removal rotating rod 608 radially arranged on the dust removal rotating shaft 602 and four dust removal driving rods 609 arranged on the dust removal base 601. The dust removal driving rods 609 are distributed around the dust removal rotating shaft 602 in a cross shape. The dust removal rotating rod 608 is provided with a magnetic block 610, and each of the dust removal driving rods 609 is provided with an electromagnet 611. Magnetic poles of the adjacent electromagnets 611 are different, and the magnetic poles of the electromagnets 611 change periodically. The magnetic block 610 and the dust removal rotating rod 608 is driven by the magnetic poles to approach or get away from the dust removal driving rods 609, so that the dust removal driving rods 609 periodically change from horizontal to vertical and then from vertical back to horizontal, thus realizing the reciprocating motion of the dust removal rotating shaft 602.

In this embodiment, the air jet holes 606 are arranged obliquely and have different degrees of inclination, so that the air discharge directions are different. The air suction holes 607 are arranged obliquely and have different degrees of inclination, so that the air suction directions are different. In combination with the rotation of the dust removal rotating shaft 602, the falling point and direction of the air jet flow, the suction point and suction direction of the air suction flow can be continuously changed to improve the dust removal effect. The dust removal rotating shaft 602 rotates periodically and reciprocatingly, and only rotates by ¼ turn for each time, so the hoses connecting the air suction fan 612 and the air jet fan 613 will not be wound together due to excessive rotation. Besides, rotation of ¼ turn is enough to constantly change the falling point and direction of the air jet flow, and the suction point and direction of the air suction flow to ensure the dust removal effect.

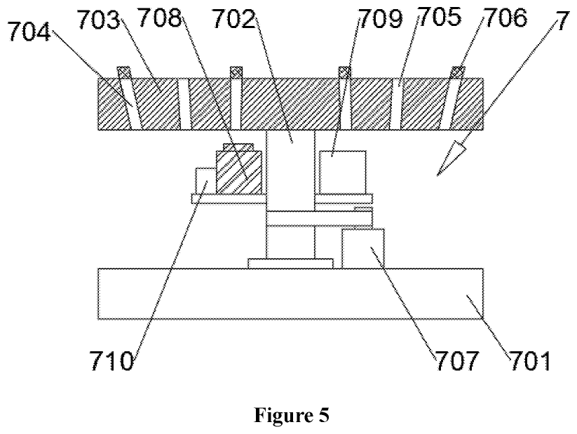

As shown in FIG. 5, the disinfection assembly 7 of this embodiment includes a disinfection base 701. The disinfection base 701 is fixed in one of the mounting grooves 12. A disinfection rotating shaft 702 is rotatably provided at the center of the disinfection base 701. The disinfection rotating shaft 702 is connected with a disinfection disc 703 which is provided with multiple disinfection holes 704 and multiple drying holes 705. The disinfection holes 704 and the drying holes 705 are arranged at intervals. A disinfection rotating motor 707 is provided on the disinfection base 701. The disinfection rotating shaft 702 is provided with a micro sprayer 708 and a drying fan 709. The disinfection rotating motor 707 is connected with the disinfection rotating shaft 702. A liquid inlet of the micro sprayer 708 is connected with a disinfectant tank 710, a liquid outlet of the micro sprayer is connected with the disinfection holes 704 through hoses. An air outlet of the drying fan 709 is connected with drying holes 705 through hoses. The disinfection holes 704 are connected with disinfection nozzles 706.

While the disinfection assembly 7 of this embodiment works, the disinfection rotating motor 707 drives the disinfection rotating shaft 702 to rotate, and the disinfection disc 703 rotates accordingly. In the meantime, the micro sprayer 708 and the drying fan 709 work, the disinfectant enters the micro sprayer 708 from the disinfectant tank 710, and is driven by the micro sprayer 708 to be sprayed out through the hoses and the disinfection nozzles 706. Then the disinfectant falls on the earphones and is dried by the airflow blown out by the drying fan 709.

In the above embodiment, the disinfection nozzles 706 are arranged obliquely with different degrees of inclination, and the drying holes 705 are arranged obliquely with different degrees of inclination, so that spraying directions of the disinfectant are different and blow directions of the air flow are different, improving the disinfection effect. Besides, the disinfectant may be alcohol, which can well remove oil stains without damaging electronic elements and can vaporize very quickly to keep the earphones dry.

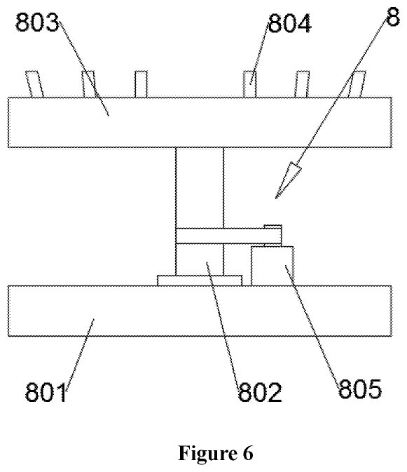

As shown in FIG. 6, the sterilization assembly 8 of this embodiment includes a sterilization base 801. The sterilization base 801 is fixed in one of the mounting grooves 12. A sterilization rotating shaft 802 is rotatably provided at the center of the sterilization base 801. The sterilization rotating shaft 802 is connected with a sterilization table 803. The sterilization table 803 is provided with multiple sterilization lamps 804 with different inclination angles. The sterilization base 801 is provided with a sterilization rotating motor 805, and the sterilization rotating motor 805 is connected with the sterilization rotating shaft 802. The sterilization rotating motor 805 drives the sterilization rotating shaft 802 to rotate, so as to rotate the sterilization table 803 and the sterilization lamps 804 thereon. The sterilization lamp 804 is a common ultraviolet lamp in the prior art, which can emit ultraviolet rays to sterilize the earphones. Since the inclination angles of the sterilization lamps 804 are different and the positions of the sterilization lamps 804 are constantly changing, the falling point and irradiation angle of ultraviolet rays are constantly changing, thereby avoiding the occurrence of sterilization dead angles and improving the sterilization effect.

In this embodiment, by providing the dust removal assembly 6, the disinfection assembly 7 and the sterilization assembly 8, the solid dust and oil stains on the earphones are first removed, eliminating the breeding ground of the bacteria. Then, the earphones are sterilized with ultraviolet rays, so that the earphones can be cleaned thoroughly.

So far, the description of the above embodiments is provided for the purpose of illustration and description. It is not intended to enumerate all possible embodiments of this disclosure or limit this disclosure. Individual elements or features of a particular embodiment are generally not limited to the particular embodiment, but when applicable, they may be interchanged and used for selected embodiments, even if without explicit illustration or description. In many aspects, the same elements or features can also be changed. This change is not considered as a departure from this disclosure, and all such modifications are included within the scope of this disclosure.

By providing exemplary embodiments, this disclosure becomes comprehensive and the scope of this disclosure is fully conveyed to those skilled in the art. In order to provide a thorough understanding of the embodiments of the present disclosure, numerous details are set forth, such as examples of specific parts, devices and methods. Obviously, it is apparent to those skilled in the art that, the exemplary embodiments may be implemented in many different forms without the specific details, and hence all of them should not be construed as limiting the scope of the present disclosure. In some exemplary embodiments, well-known processes, well-known device structures, and well-known technologies are not described in detail.

Professional terms are used only for the purpose of describing specific exemplary embodiments, and are not meant for limitation. Unless the context clearly indicates otherwise, the singular forms “a” and “the” as used herein may further include the plural form. The terms “including” and “having” are inclusive, and specify the existence of the stated features, entities, steps, operations, elements, and/or assemblies, but do not exclude the presence or additional inclusion of one or more other features, entities, steps, operations, elements, assemblies and/or combinations thereof. Unless the sequence of execution is explicitly indicated, the method steps, processes and operations described herein do not necessitate to be executed in the specific sequence described and illustrated. It should also be understood that additional or alternative steps may be taken.

If an element or layer is referred to as being “on”, “bonded with”, “connected to” or “coupled to” another element or layer, it may be directly on, bonded with, connected to or coupled to another element or layer, or there may be elements or layers intervened therebetween. In contrast, if an element or layer is referred to as being “directly on”, “directly bonded with”, “directly connected to” or “directly coupled to” another element or layer, there may be no element or layer intervened therebetween. Other terms for describing relationships among the elements should be interpreted in a similar manner (for example, “between” and “directly between”, “adjacent” and “directly adjacent”, etc.). The term, “and/or”, as used herein includes any one of all possible combinations of one or more of the associated listed items. Although the terms, “first”, “second”, “third” and the like, may be used herein to describe various elements, assemblies, regions, layers and/or portions, these elements, assemblies, regions, layers and/or portions are not limited by these terms. These terms may only be used to distinguish one element, assembly, region or portion from another element, assembly, region or portion. Unless clearly indicated by the context, the use of terms such as “first”, “second”, and other numerical values herein does not imply a certain sequence or order. Therefore, the terms, first element, assembly, region, layer or portion, discussed below may be referred to as second element, assembly, region, layer or portion without departing from the teachings of this exemplary embodiment.

Terms of space relative relationship, such as “inside”, “outside”, “below”, “under”, “lower”, “above”, and “upper”, may be used here for convenience of description to describe the relationship between one element or feature and another one or more elements or features as shown in the figure. Terms of space relative relationship may not exclude that the elements or features may have different orientations in addition to those orientations depicted in the figure. For example, if the device in the figure is turned over, elements described as “below other elements or features” or “under elements or features” will be oriented as “above other elements or features”. Therefore, the exemplary term “under” may include both upward and downward orientations. The device may be oriented in other ways (for example, being rotated by 90 degrees or oriented in other directions) and described in terms of the space relative relationships of the new orientations.

1. An earphone storage device, comprising a charging storage box (1), wherein a top cover (2) that is closable is provided on the charging storage box (1), a storage tank (3) and a cleaning device (4) are provided in the charging storage box (1), a charging assembly (5) is provided at the bottom of the storage tank (3), and the cleaning device (4) comprises a dust removal assembly (6), a disinfection assembly (7) and a sterilization assembly (8); and
a rotating shaft (9) and a rotating motor (10) are rotatably provided at the center of the top cover (2), the rotating motor (10) is connected with the rotating shaft (9), the rotating shaft (9) is connected with a rotating disc (11), the rotating disc (11) is provided with three mounting grooves (12), the dust removal assembly (6), the disinfection assembly (7) and the sterilization assembly (8) are respectively fixed in the three mounting grooves (12), and when the top cover (2) is closed, the storage tank (3) is located directly below a rotation track of the mounting groove (12).

2. The earphone storage device according to claim 1, wherein the dust removal assembly (6) comprises a dust removal base (601), a dust removal rotating shaft (602) is rotatably provided at the center of the dust removal base (601), the dust removal rotating shaft (602) is connected with a dust removal disc (603), a plurality of dust removal strips (604) are radially arranged on an upper surface of the dust removal disc (603), each of the dust removal strips (604) is provided with a dust removal brush (605), the dust removal disc (603) is provided with a plurality of air jet holes (606) and a plurality of air suction holes (607), and the air jet holes (606) and the air suction holes (607) are arranged at intervals.

3. The earphone storage device according to claim 2, wherein the air jet holes (606) are arranged obliquely and have different degrees of inclination, and air discharge directions of the air jet holes are different; and the air suction holes (607) are arranged obliquely and have different degrees of inclination, and air suction directions of the air suction holes are different.

4. The earphone storage device according to claim 3, wherein the dust removal rotating shaft (602) is radially provided with a dust removal rotating rod (608), the dust removal base (601) is provided with four dust removal driving rods (609), the four dust removal driving rods (609) are distributed around the dust removal rotating shaft (602) in a cross shape, the dust removal rotating rod (608) is provided with a magnetic block (610), and each of the dust removal driving rods (609) is provided with an electromagnet (611).

5. The earphone storage device according to claim 4, wherein the dust removal base (601) is provided with an air suction fan (612) and an air jet fan (613), a dust collecting box (614) is connected to an air outlet of the air suction fan (612), an air inlet of the air suction fan (612) is connected to the air suction holes (607) through hoses, and an air outlet of the air jet fan (613) is connected to the air jet holes (606) through hoses.

6. The earphone storage device according to claim 1, wherein the disinfection assembly (7) comprises a disinfection base (701), a disinfection rotating shaft (702) is rotatably provided at the center of the disinfection base (701), the disinfection rotating shaft (702) is connected with a disinfection disc (703), the disinfection disc (703) is provided with a plurality of disinfection holes (704) and a plurality of drying holes (705), and the disinfection holes (704) and the drying holes (705) are arranged at intervals.

7. The earphone storage device according to claim 6, wherein the plurality of disinfection holes (704) are connected with disinfection nozzles (706), the disinfection nozzles (706) are arranged obliquely and have different degrees of inclination, spraying directions of the disinfection nozzles are different, the drying holes (705) are arranged obliquely and have different degrees of inclination, and air discharge directions of the drying holes are different.

8. The earphone storage device according to claim 7, wherein a disinfection rotating motor (707) is provided on the disinfection base (701), the disinfection rotating shaft (702) is provided with a micro sprayer (708) and a drying fan (709), the disinfection rotating motor (707) is connected with the disinfection rotating shaft (702), a liquid inlet of the micro sprayer (708) is connected with a disinfectant tank (710), a liquid outlet of the micro sprayer is connected with the disinfection holes (704) through hoses, and an air outlet of the drying fan (709) is connected with the drying holes (705) through hoses.

9. The earphone storage device according to claim 1, wherein the sterilization assembly (8) comprises a sterilization base (801), a sterilization rotating shaft (802) is rotatably provided at the center of the sterilization base (801), the sterilization rotating shaft (802) is connected with a sterilization table (803), the sterilization table (803) is provided with a plurality of sterilization lamps (804) with different inclination angles, the sterilization base (801) is provided with a sterilization rotating motor (805), and the sterilization rotating motor (805) is connected with the sterilization rotating shaft (802).

10. The earphone storage device according to claim 1, wherein a central control device (13) is further provided in the storage box (1), the charging assembly (5) and the cleaning device (4) are both connected with the central control device (13), and after the charging assembly (5) starts working, the central control device (13) drives the cleaning device (4) to operate.
